(19) United States
(12) Patent Application Publication
(10) Pub. No.: US 2008/0051833 A1
(43) Pub. Date: Feb. 28, 2008

(54) Suture passer and method of passing suture material

(76) Inventors: Vincent Gramuglia, Scarsdale, NY (US); Martin Wendelken, Elmwood Park, NJ (US)

Correspondence Address: VINCENT GRAMUGLIA, 104 VERNON DRIVE, SCARSDALE, NY, 10583, US

(21) Appl. No.: 11/509,882

(22) Filed: Aug. 25, 2006

(51) Int. Cl. A61B 17/06

(52) U.S. Cl. 606/222

(57) ABSTRACT

A suture and needle combination is disclosed for use during surgical and non-surgical procedures comprising a suture needle 16 with a breakaway tip 20, insertable tip 24 or reusable tip 32. Suture 10 passes freely through hollow needle 16 after a user removes needle tip (20,24, or 32) during use. This suture needle combination reduces the amount of room needed within a surgical site, reduces the length of needle 16 needed to exit tissue (for example) while suturing, and decreased trauma to at risk anatomical structures during a surgical procedure or other use.

### FIELD OF THE INVENTION

This patent relates to a suture passer and a method of passing suture for use in non-medical and medical situations including both open and minimal incision surgical procedures.

### BRIEF DESCRIPTION OF PRIOR ART AND BACKGROUND F THE INVENTION

In medicine, suture is passed through soft tissue and bone in many ways. Suture material is used in human and veterinary medicine for many reasons. Some examples are the closure of a wound by approximating the skin margins surrounding the wound. Also suture is used to bring together subcutaneous tissues that have been dissected during surgical procedures. Still, other uses of suture material in medicine include a method to close vessels by tying a loop around a ligated or patent vessel, the repair of torn tendons, and the reattaching a tendon that has been avulsed from its insertion on bone. Suture material can be classified as either absorbable or non-absorbable. Absorbable suture is usually placed below the skin surface where in time, the body decomposes, dissolves, and absorbs the suture material. There are numerous non-absorbable suture materials also used during surgical procedures. The non-absorbable materials are usually employed and manually removed after the intended purpose has been completed such as a surgical site that is considered healed.

The most commonly taught method of using suture material during formal education involves pushing of a sharp end of a needle through tissue. This same needle has on its opposite end a length of suture material attached which is pulled along with the needle through the tissue. This method of using a suture & needle combination comprises grasping the body of the sharp needle (mid shaft) with a forceps or a clamp. The sharp leading end of the needle is then pushed the through the tissue until the leading end exits the tissue and is exposed. A physician will then grasp the exposed leading end of the needle a second time with the forceps or clamp. The suture & needle combination is then pulled partially (using the forceps or clamp) out of the tissue leaving some of the attached suture within the tissue. The suture is then tied using any one of a number surgical knots to approximate, close, ligate, or attach structures depending on which of the various uses the physician is trying to accomplish.

Often while utilizing a suture & needle combination, it is difficult to grasp and pull the entire needle through the tissue. This common situation is usually due to the suture needle being too large for the space provided by a particular incision site. In addition to the limited space available by incision sites, the size, shape, and or rigidity of the suture needle utilized may also be self-limiting during the surgical procedure. Additionally, one may be unable to pull the needle through tissues due to the proximity of anatomical structures which are at risk of injury caused by the suture needle.

Primarily, suture and needle combinations are most often thought to be in medicine (human and veterinary), however many other industries use a suture and needle combination including upholstery, shoemaking, and tailors (clothing repair).

Some prior art does address some of these needs by providing various new methods of passing suture. One example is U. S. Patent Application No. 2005/0182446 (2005), DeSantis; who employs a needle suture combination where the leading end of the suture needle (usually sharp) has a spherical bunt tip. This bunt tip is passed through tissue and provides some protection to at risk structures during surgical repair using a suture & needle combination. Another suture & needle combination to aid in the passing of suture through tissues was disclosed in U. S. Patent Application No. 2005/0096698 (May 2005) Lederman, who teaches of a suture needle employing visual indicators using colors. The needle colors are intended to allow for increased needle visualization and orientation when exiting tissue. Still other improvements are suggested to suture needles in U.S Patent Applications 2004/0059380 (March 2004), 2004/0098048 (May 2004) and 2004/0106948 (June 2004) all by Cunninghan. Here the applicant concentrates on the distal leading edge of the suture needle suggesting a variety of needle surfaces to enhance the passing of the needle through tissue. Although the above stated improvements for passing suture through tissue are novel for their particular applications including endoscopic uses, none of the patents or applications teach a novel method that reduces needle size to prevent injury to local tissues. Further, none of the disclosures teach about suture & needle combinations that employ size reduction for use in small incision sites. There are a number of patented devices describe in prior art for passing suture material during endoscopic surgical procedures. U.S. Pat. No. 6,984,237 Hatch, et. al. January 2006, describes a surgical instrument that provides stabilization of tissue while suture is passed. Although this device does provide a solution for its intended purpose, it is a complex and an expensive solution for passing suture. Further, it appears that the Hatch device appears to be best utilized in remote endoscopic surgical procedures. Presently in medicine, there are numerous scenarios where surgeons today are sill in need of simple solution to allow suture to be passed through tissue within a small opening in the body. Such applications are not necessarily an endoscopic surgical procedure but a procedure performed through a small opening in the skin. The above disclosed patents and patent applications are novel for their intended purposes. However, none of the above inventions provides solution that describes a suture & needle combination that provides needle size reduction during use. The prior art also fails to disclosed or illustrate a suture & needle combination with a removable or break away tip at its distal end having suture material that remains attached. In another disclosed U.S. Pat. No. 3,949,756; Ace, 1976, a suture & needle combination is described as having a weakened segment within the suture material itself. This weakened segment permits a surgeon to separate the suture from the needle using a forceful tug of the suture while holding the suture needle. This combination is used to eliminate the need of having to employ scissors to cut the suture material. Still other patents describe suture & needle combinations like U.S. Pat. No. 4,054,144; Hoffman, et. al. 1977. Hoffman's patent unlike the Ace disclosure is focused on providing increased strength of the attachment of suture material to the needle. These improvements to suture & needle combinations are novel for their intended purpose but fail to describe or provide a suture & needle combination with a break away or removable tip. None of the above disclosures allows for a small part of a needle to be exposed within a surgical field, which allows a surgeon to dislodge this exposed needle portion from the main body of a suture needle. Further, none of the prior art describes a breakaway needle tip having the suture material attached for uses in small surgical sites. A suture needle with break away or removable tip is easily grasped within a small surgical field allowing physicians easily pass the suture material through tissue. This effective solution will provide surgeons with the ability to pass suture material in a small surgical field while avoiding damage to adjacent anatomical structures. These benefits along with other advantages over prior art will become apparent after reading the following description and viewing the provided drawings described herein.

### BRIEF DESCRIPTION OF THE DRAWINGS

FIG. 1a shows a conventional straight suture & needle combination having a pointed sharp tipped needle on one end with a length of suture material attached to the opposite end.

FIG. 1b shows a conventional suture & curved needle combination having a pointed sharp tipped curved needle on one end with a length of suture material attached to the opposite end.

FIG. 2a illustrates two views of a straight hollow needle & suture combination. This needle has a notch made in the needle shaft located a short distance from the sharpened end providing a breakaway needle tip. These two images show the needle together and then with the break away tip exploded.

FIG. 2b illustrates two views of a curved hollow needle with a length of suture material attached. This needle has a notch made in the needle shaft located a short distance from the sharpened end providing a breakaway needle tip. These two images show the needle together and then the needle with the break away tip exploded.

FIG. 3a-b shows a hollow needle & suture combination illustrating a removable needle tip both assembled (3a) and exploded (3b).

FIG. 4a-e shows a needle & suture combination which uses a reusable removable needle tip. Steps 4a-e illustrate how the reusable needle may be re-threaded and reassembled for a second use.

FIG. 5a-c show a number of variations of a needle & suture combination having a removable needle tip and a needle shaft comprising a reservoir that holds a length of suture within. 5-a has length of folded suture, 5-b shows suture wound about a tube, and 5-c shows suture material stored wound about a spool.

FIG. 6a-e illustrates the steps and use of a straight needle & suture combination with a breakaway tip passing suture through a bone and tying a tendon to the outer surface of bone.

FIG. 7a-d illustrates the steps and use of a curved needle & suture combination with breakaway tip passing suture through soft tissue or other similar structure.

FIG. 8a-c shows the use of a curved needle & suture combination with breakaway tip being used to pass suture through a bone within an open surgical incision. The suture is then used to secure a tendon to the bone.

### REFERENCE NUMERALS IN DRAWINGS

| | |
|---|---|
| 10 Suture Material | 12 Conventional Straight Suture |
| | Needle |
| 14 Conventional Curved Suture Needle | 16 Suture Needle Shaft with |
| | notch |
| 18 Open end in Suture Needle | 20 Breakaway Suture Needle tip |
| 22 Notch in Suture Needle | 24 Insertable Needle Tip |
| 26 Tapered Needle Shaft | 28 Body of Insertable Needle |
| 30 Reusable Tapered Needle Shaft | 32 Reusable Needle Tip |
| 34 Suture remnant | 36 Suture Reservoir |
| 38 Suture wound on cylinder | 40 Bone |
| 42 Hole in Bone | 44 Suture wound on spool |
| 46 Tendon | 48 Clamp |
| 50 Tissue Sample | 52 Suture Knot |

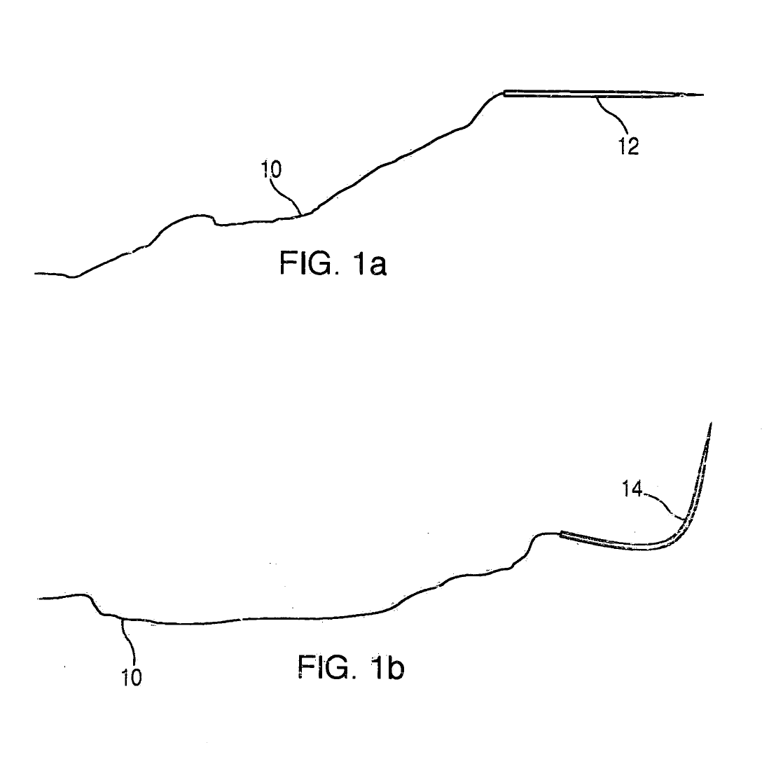

### Overall Description of Embodiment—FIGS. 1 to 8

Directing attention to FIG. 1a-b of the drawings, two images of a suture & needle combination. Typically, suture & needle combination (FIG. 1a) has a suture needle 12 that is straight and has one end that is sharp. On the opposite end of needle 12 suture material 10 is directly attached in any one of a numbers of ways to straight suture needle 12. In FIG. 1b, a suture & needle combination is shown having a curved suture needle 14 with suture material 10 attached in a similar fashion as seen and described in FIG. 1a.

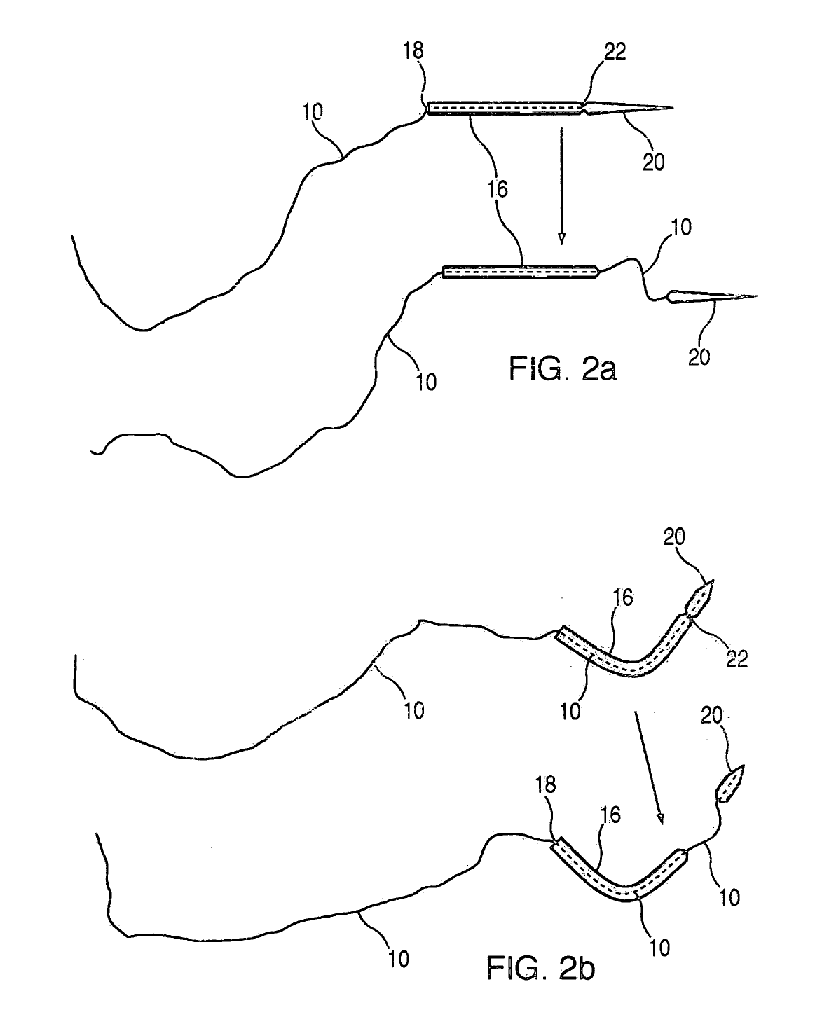

In FIG. 2a-b, two more suture & needle combinations are shown. In FIG. 2a straight suture needle shaft 16 is depicted having two ends, a sharp breakaway tip 20 on one end and the second end which has suture material 10 emanating from an opening 18 in straight suture needle shaft 16. Straight needle shaft 16 also has a notch 22 that has been made near the distal sharp end of straight needle shaft 16. Notch 22 provides a means to weaken straight needle shaft 16 allowing for separation of breakaway tip 20. In addition FIG. 2a shows that straight needle shaft 16 is hollow which allows suture material 10 to traverse the shaft of straight needle shaft 16. In FIG. 2a, a second image is shown with breakaway tip 20 having been separated from straight suture needle shaft 16. Note that suture material 10 is still attached to breakaway tip 20 as it is moved away from straight needle shaft 16. Opening 18 in straight needle shaft 16 allows suture material 10 to be pulled through hollow suture needle shaft 16. In FIG. 2b illustrates a curved needle shaft 16, which also has an opening 18 on one end, a notch 22 a short distance form the sharp end of breakaway tip 20.

In FIG. 2b, an illustration of suture 10 and curved needle shaft 16 combination is depicted first as one complete unit and a second drawing in FIG. 2b there is a curved needle shaft 16 having breakaway tip 20 distracted at notch 22 which provides a means to separate breakaway tip 20 along with suture 10 still attached.

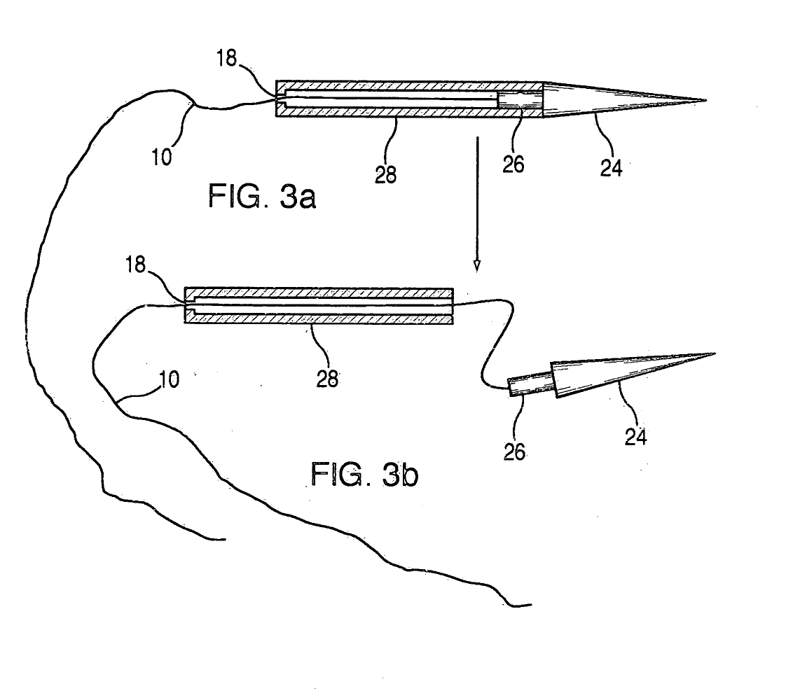

In FIG. 3a-b another suture needle combination is presented. In FIG. 3a, suture 10 again is depicted entering the body of needle 28 through opening 18 and traverses the entire shaft of needle 28. Suture 10 attaches to an insert shaft 26, which is part of removable needle 24. FIG. 3b, shows the removable needle 24 after being extracted from needle body 28 having suture 10 attach to needle 24 on the end of the insert shaft 26.

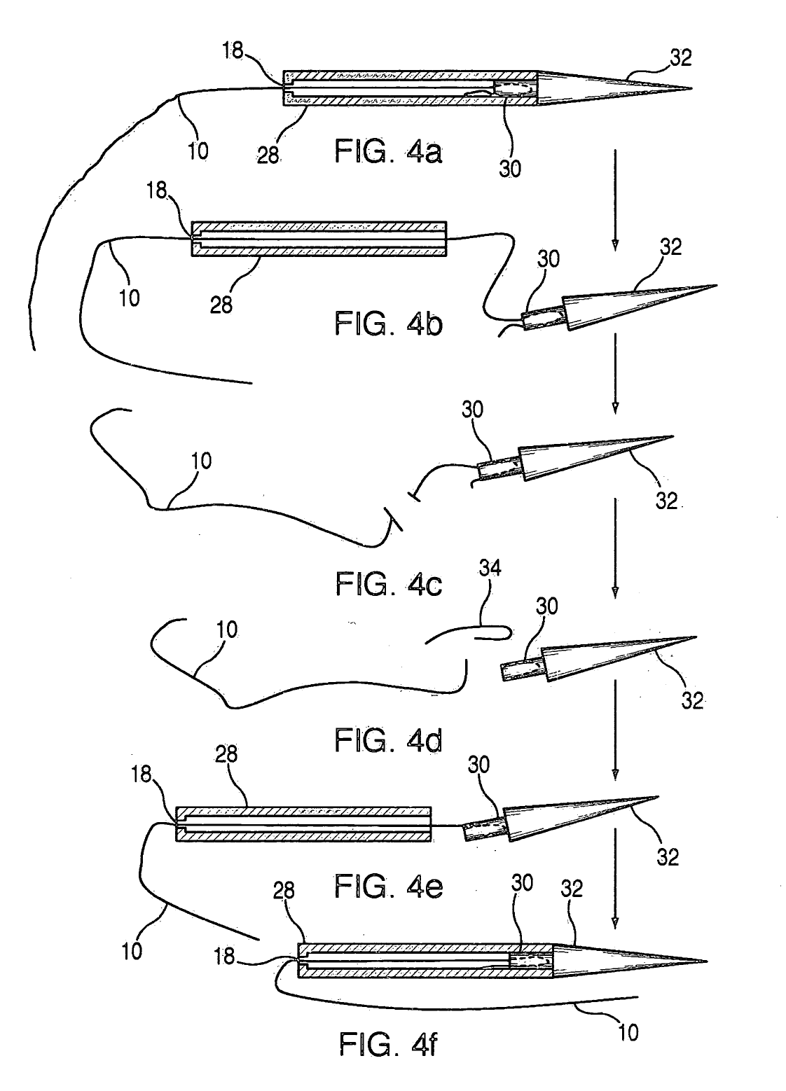

In FIG. 4a-e, a suture neelde combination is shown that employs both a resuable needle 32 and suture 10. In FIG. 4a, when assembed resuable needle 32 and associated needle insert 30 is placed within needle shaft 28. Suture 10 is attached to needle insert 30 and travel through needle shaft 28 and exits through opening 18. Note that suture 10 attachment to needle insert 30 is looped and therefore is removable.

In FIGS. 4a-f, a series of pictures that reveals the abilty to reuse suture 10, needle 24, and shaft 28. Starting with FIG. 4a, the needle & suture combination is assembled. FIG. 4b, shows resuable needle 32 with suture 10 attached, now distracted from needle shaft 28, FIG. 4c, shows that suture 10 is cut; FIG. 4d illustrates the removal of suture remenant 34 from needle insert 30; followed by FIG. 4e, re-attachment of suture 10 to removable needle 32 insert shaft 30; and lastly, in FIG. 4f, re-assembly of reusable the suture & needle combination.

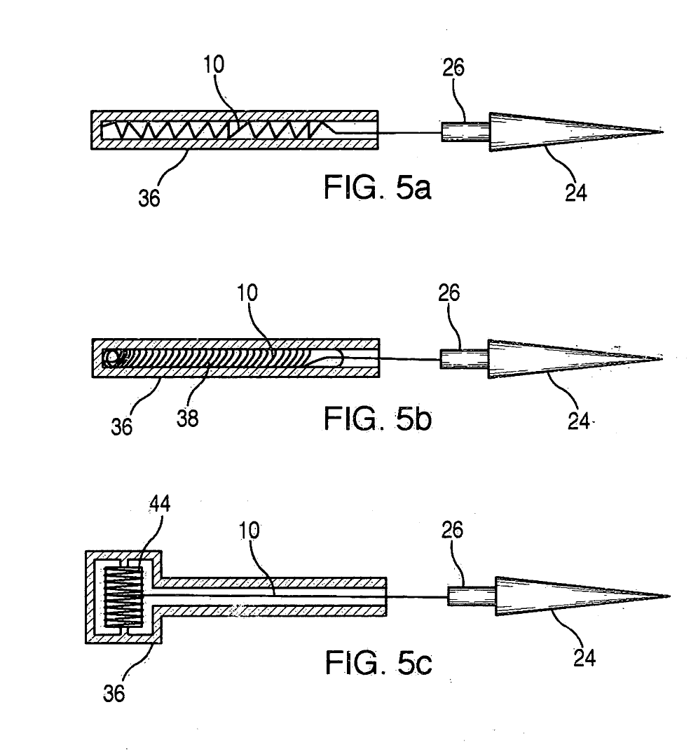

FIG. 5a-c is shown another configuration of a suture needle combination as follow: Suture needle 24 is pictured as a removable needle 24 type with an insertable needle shaft 26. Suture 10 is contained within a reservoir 36, which provides a means for storing various lengths of suture material 10. A variety of methods of storing suture material 10 are possible and 3 examples are shown. FIG. 5a, shows suture material 10 in a folded configuration within reservoir 36, while in FIG. 5b, suture 10 is wound about a cylinder 38 contained within reservoir 36, and lastly, in FIG. 5c, suture 10 is stored on a spool 40 within reservoir 36.

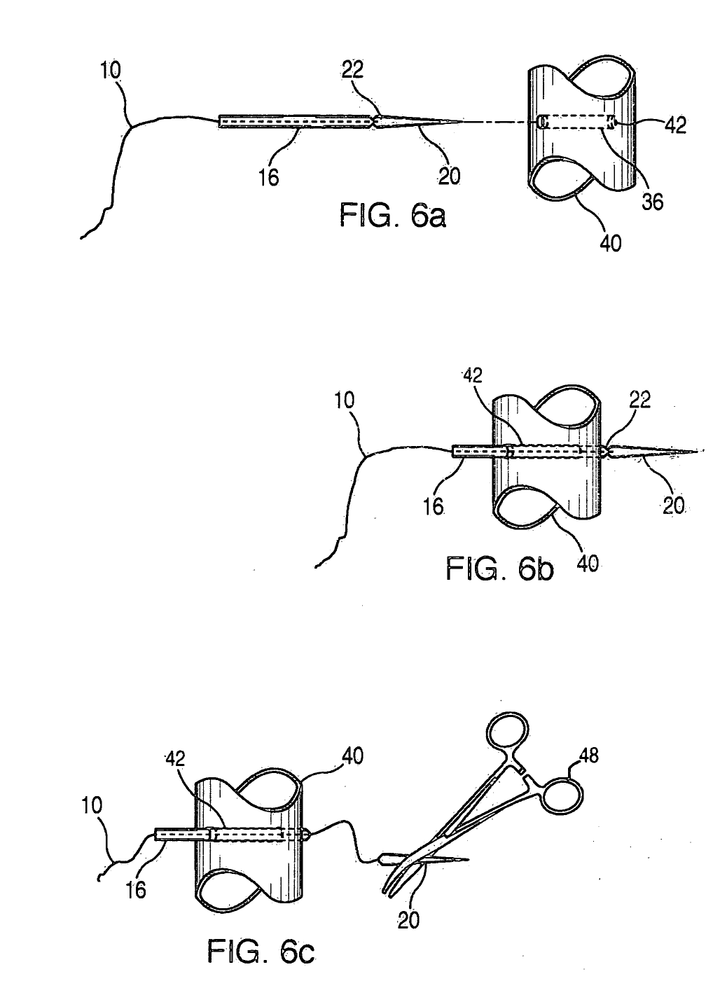

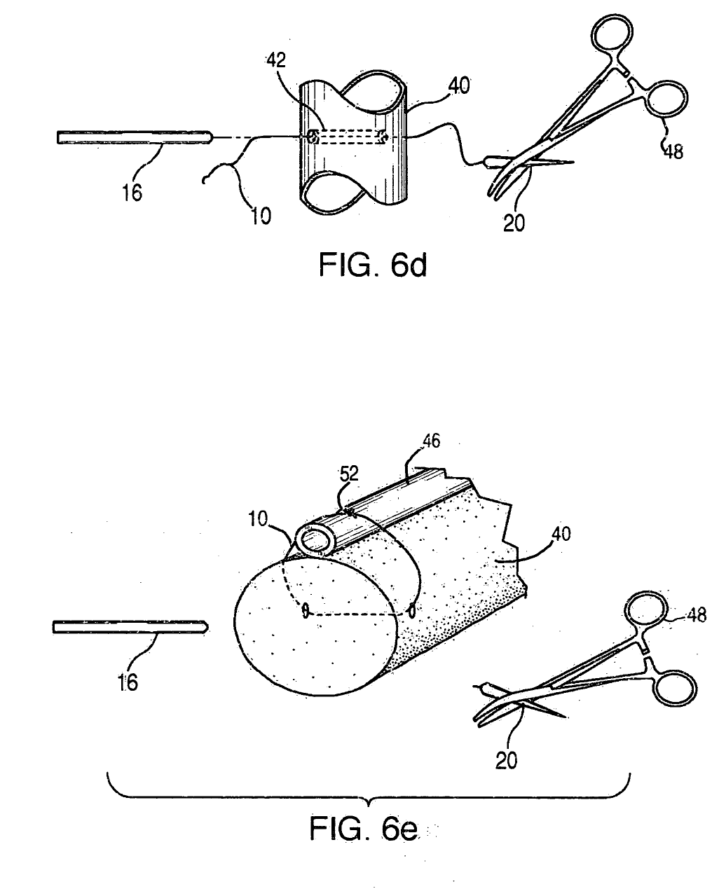

In FIG. 6a-e a suture & needle combintaion with breakaway tip 20 is shown in steps tying a closed loop of suture mateiral 10 through an opening 42 in a bone 40 and around tendon 46. In step 6a illustrates a straight suture needle shaft 16 aligned to be passed through opening 42 in bone 40. Step 6b the suture needle shaft 16 is pushed through bone 40 just until breakaway tip 20 is exposed showing notch 22 exiting from bone 40. Step 6c shows breakaway tip 20 grasped with clamp 48 now distracted form needle shaft 16 with suture material 10 attached and moved a distance away from bone 40. Step 6d, illustrates the remaining straight needle shaft 16 now dislodged from breakaway tip 20 being removed from opening 42 in bone 40. This leaves suture material 10 within opening 42 in bone 40. Lastly, FIG. 6e illustrates a loop of suture material 10 (that includes tendon 46) which is used to hold tendon 46 in place using surgical knot 52 against the outer surface of bone 40.

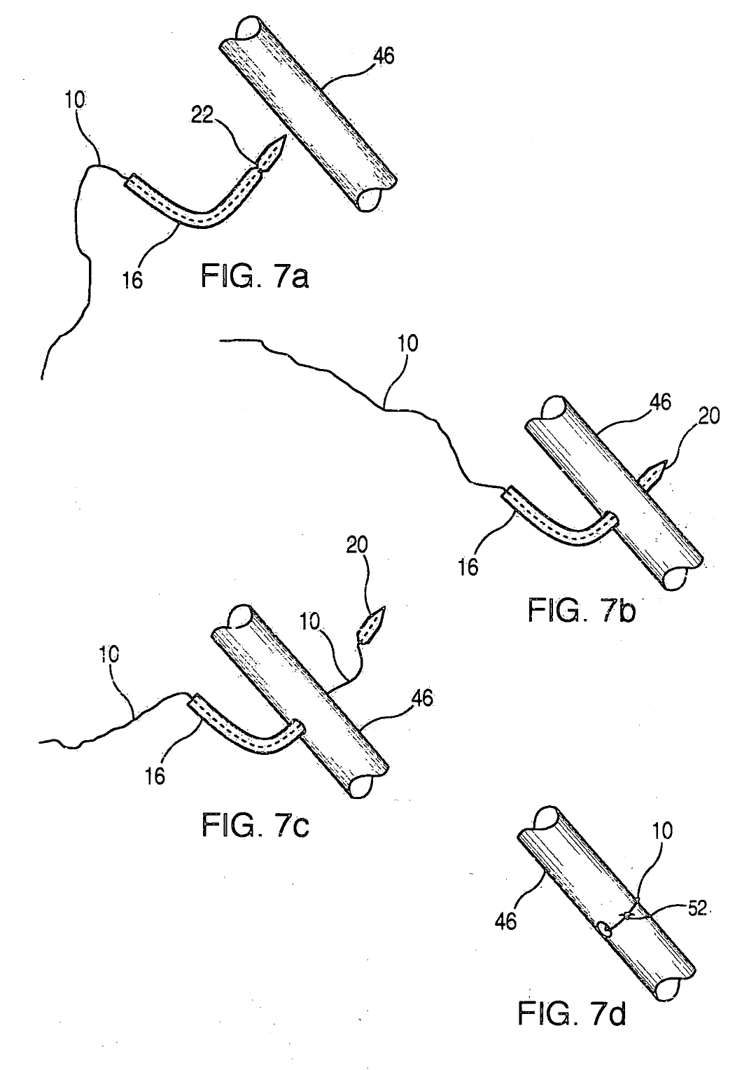

FIG. 7a-d shows the steps made when using a curved suture needle shaft 16 with notch 22 and breakaway tip 20 used to pass suture 10 through a tendon 46 or other type of soft tissue. FIG. 7a begins with a intact tendon 46 and curve suture needle 16 prior to being inserted into tendon 46. FIG. 7b the curved suture needle 16 has been pushed through tendon 46 just enough to expose breakaway tip 20. In FIG. 7c, breakaway tip 20 has been disloged from curved needle shaft 16 having suture material 10 attached to breakaway tip 20. In FIG. 7d, the remaining needle shaft 16 has been remove leaving suture materal 10 within allowing for the completion of tying surgical knot 52.

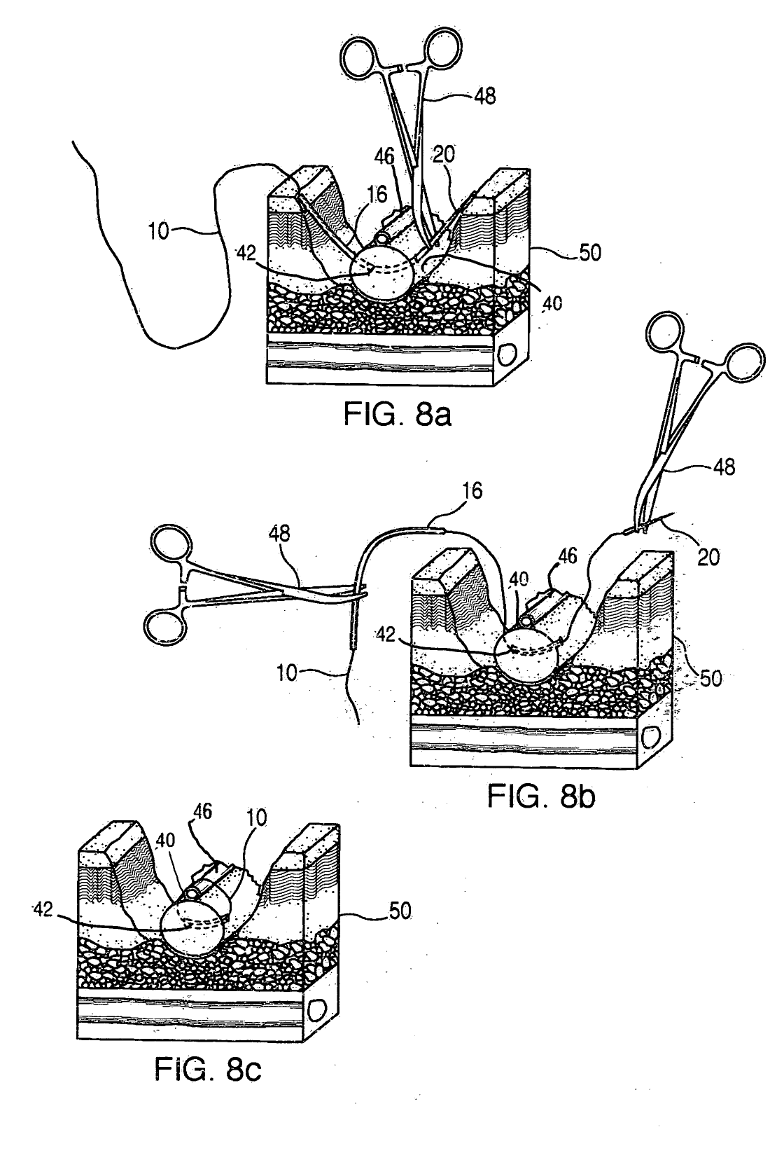

FIG. 8a-c shows the steps of using the novel suture & needle combination within an incsion site. FIG. 8a is a segment of soft tissue 50, with incision is depicted having a bone 40 and a loose tendon 46 contained within. Suture 10 and a curved needle having a shaft 16 and a breakaway tip 20 have been passed through a transverse hole 42 in the shaft of bone 40. FIG. 8b shows breakaway tip 20 with suture 10 attached grasped by clamp 48, now extracted from the tissue 50. Needle shaft 16 has also been removed from bone 40 and tissue 50 leaving the opposite end of suture 10 within bone 40 and tissue 50 incision site. In FIG. 8c, suture 10 is shown being used to approximate tendon 46 to the outer surface of bone 40.

### Operation Of The Invention—FIGS. 1, 2, 3, 4, 5, 6, 7, 8

FIG. 1a-b Conventional suture 10 and needle 12 combination are commonly use in surgical procedures. Needle 12 is typically made of stainless steel and may vary in lengh and shape. Straight needle 12 has applications in certain surgical procedures, while needle 14 for example illustrates a curved shape suture needle combination which may be perferred in other procedures. The method of attachment of suture 10 to needles 12, 14 varies but the vast majority involves pinching a small length of suture 10 within a small recess on the dull end of needles 12,14. Needles 10, 12 are pushed through various tissues along with suture 10 trailing within the same path. A major problem presented by conventional suture needle combinations is the risk of injury to local tissues and anatomical structures during use.

FIG. 2a-b show two novel suture needle combination that provide a solution to reduce injury to local tissues and anatomical structures. The needle suture combination in FIG. 2a has a needle 16 that is linear. Needle 16 starting at open end 18 is hollow. Suture 10 enters the shaft of needle 16 and travel the length of needle 16 past notch 22 and into breakaway tip 20. Suture 10 is attached to breakaway tip 20 in any number of methods including adhesives or other mechanical means. FIG. 2a has a second image that illustrates the same suture needle combination now having breakaway tip 20 seperated from needle shaft 16 at notch 22. Notch 22 provides a weakend point along the shaft of needle 16 making it easy to separate breakaway tip 20 from needle shaft 16. As breakaway tip 20 is moved away from needle shaft 16, suture 10 freely flows through the hollow needle shaft 16. FIG. 2b shows a similar suture needle combination having a hollow needle shaft 16 with open end 18, notch 22, and breakaway tip 20 and differs only in that needle 16 is now is curved. Notch 22 in combination with breakaway tip 20 offers many advantages over previous art. Needle suture combinations as illustrated in FIGS. 2a-b may be of any length and have curves of any number of angles. In practice a surgion using a suture needle combination employing a hollow shaft needle 16 with notch 22 and breakaway tip 20 has the advantage of not having to pull the entire needle 16 through tissue to pass suture 10. A physician need only expose breakaway tip 20, dislodge breakaway tip 20 from needle 16 at notch 22. While holding breakaway tip 20, the physician then withdraws needle shaft 16 in the opposite direction of the original path of entry, and disposes needle shaft 16. Suture 10 remain within the tissue path allowing for the completion and tying of suture 10 into surgical knots. An actual example is issustrated in drawings that follow.

Direction attention to FIGS. 3a-b where images illustrate another suture needle combination. FIG. 3a shows a needle shaft 28 that has an opening 18 on one end, with suture 10, that enters and travels the length of needle shaft 28. Suture 10 attaches to an insertable needle tip 26 which is a tapered section of an insertable needle tip 24 which then will fit within the shaft of needle 28. Needle 28 has the same advantages as the examples shown in FIG. 2a-b and differs in that there is no notch 22 (FIG. 2a-b) in needle shaft 28. Needle 28 exploys an insertable needle tip 24 that has a tapered needle shaft 26. Tapered needle shaft 26 is not perminently fixed providing a means for removing the needle tip 24 from needle 28. When using needle 28 and suture 10 during a surgical procedure, a physican has the same advantages, that being, the ability to grasp the removable needle tip 24 without having to push the entire needle shaft 28 through the tissue. Variations of needle tip 24 and tapered shaft 26 attachment to needle shaft 28 may include a variety of methods such as a twist lock, threaded screw, or snap on needle tip.

FIG. 4a-e illustrates a reuseable needle & suture combination. FIG. 4 employs a similar type of removable needle 32 that is inserted in the saft of needle 28. However, the insertable portion of needle 32, the tapered section 30, allows for the reattachment of suture 10 to needle tip 32. The sequence of reusing same needle tip 32 in incombination with needle shaft 28, and suture 10 is as follows: FIG. 4a needle 28 with reusable tip 32 having suture 10 attached is pictured assembled as a single unit. FIG. 4b removable tip 32 is extracted from needle shaft 28. Suture 10 is then cut (FIG. 4c). In FIG. 4-d suture remnant 34 is removed from tapered attachment site 30. Suture 10 (FIG. 4-e) is then reattached by looping or clamping suture 10 to tapered attachment site 30. Suture 10 is reintroduced and pass through needle shaft 28 and needle tip 32 is then reinserted into needle shaft 28. Needle tip 32 having been reinserted into needle shaft 28 (FIG. 4F) provides a novel reusable needle & suture combination. Again variations on how needle tip 32 and tapered attachment site 30 is removabaly attached to needle shaft 28 may include a variety of methods such as a twist lock, threaded screw, or snap on needle tip.

In FIG. 5a-c depicts other suture needle combinations each of which has a reservior 36 wich serves as a needle shaft while also being used to house a length of suture 10 within. FIG. 5a needle shaft reservior 36 is shows a length of suture 10 that has been folded within reservior 36. Storing suture 10 within a sealed reservior 36 provides additional benefits for user including increased sterility and easier management and handling of suture material 10. FIG. 5b shows suture 10 wound about a cylinder 38, and FIG. 5c has suture 10 stored upon a spool 40 within reservior 36. Needle reservior 36 as shown illustrates a removiable needle tip 24 however, such needle & suture combinations may also employ a breakaway tip 20 or reusable needle tip 32.

FIGS. 6a-e show the steps of using a suture & needle combination with a breakaway tip 20 to tie a tendon 46 structure to the outer surface of bone 40. FIG. 6a begins with bone 40 that has a hole 42 that run the entire width of bone 40 and a straight suture needle 18, with notch 22 and breakaway tip 20. A physician will place straight suture needle 18 into one opening 42 in bone 40 and push needle 18 until breakaway tip 20 exit opening 42 on the oppsite side of bone 40. (FIG. 6b). Next clamp 48 is used to grasp and breakaway tip 20 (FIG. 6c) from needle shaft 16 pulling suture 10 along with tip 20. In FIG. 6d needle shaft 16 is withdrawn from bone 40 exposing the remaining suture material 10 outside of bone 40. FIG. 6e a loop of suture 10 is used to approximate soft tissue tendon 46 to the outside surface of bone 40. The advantages of not having to pull the entire shaft of needle 16 should not be under stated. This novel suture & needle combination reduces costs, time, and materials when compared to other procedures to approximate a tendon to bone. Similar results may be obtained using expensve hardware and equipment such as a surgical screw being place through tendon 46 and into the cortical surface of bone 40. Such procedures using hardware increase risks to patients that include foreign body reactions and a higher probabality of infection due to time needed to perform the procedure.

A great advantage of this novel suture & needle combination is the use of a curved needle shaft 16 to suture a tendon 46 as depicted in drawings FIG. 7a-d. Here the needle 16 is pushed through a tendon 46 (7b) just enough to expose tip 20. Breakaway tip 20 is dislodged (7c) from needle shaft 16 and a physician then completes the procedure by tying suture 10 using surgical knot 52. It sould be clear that not having to pull an entire needle through anatomical structures has many advantages over prior art.

This can best be illustrated in FIGS. 8a-c where the use of this novel suture & needle combination occures within an incision site in tissue 50. Limited space within incision sites many times require that surgions make wounds larger and deeper to allow suturing to be performed. Larger incision sites allow for better visualization and room to work, however greatly increase healing time, post operative pain, and risk of infections. Suture needle 16 with breakaway tip 20 reduces the space needed to perform a surgical procedure. After placing suture needle 16 through opening 42 in bone 40, (FIG. 8a), clamp 48 is used to remove breakaway tip 20 from needle shaft 16 (FIG. 8b) while remaining needle shaft 16 is also removed from the inscion site in tissue 50. Tendon 46 is easily approximated to the outer surface of bone 40 by looping and tying suture 10 (FIG. 8c). This procedure greatly avoids injury to local structures that may be within or around the incision site in tissue 50.

### SUMMARY AND SCOPE

After reading the fore stated description about this novel “Suture Passer and Method of Passing Suture Material” one realizes that this simple solution provides many benefits to both physicians and patients.

Some advantages are:
The ability of suture needle 16 with a breakaway tip 20 to allow physicians to work in smaller incision sites.
The reduction of injury to local tissue 50 and the decrease risk to vital structures within or near surgical site while performing procedures.
This suture 10 & needle 16 combination with breakaway tip 20 also provides a lower cost alternative to using expensive hardware, surgical screws, and other implants for utilized in the surgical procedures.
This novel invention will reduce the risk of infection through smaller surgical openings while in addition to decrease exposure due to reduced operating time needed when performing procedures.
This invention provides a method for having the ability to utilize re-useable suture needle tips 32 again reducing costs even further.

An additional benefit is that this invention does not require any new skills to take advantages of the increased safety, reduced risk of infection, and time saving offered by this novel suture and needle combination. Even further, there are no special accessory tool or additional energy requirements needed to use this invention.

While the invention has been explained by a detailed description of certain specific embodiments and has focused on medicine (human and veterinary) applications, there are many other industries including upholstery, shoemaking, and tailors (clothing repair) for example, which also will benefit from this invention. It is understood that in other areas of industry, various modifications and substitutions can be made. These variations should be included within the scope of the appended claims, and also should include the equivalents of such embodiments.

Having described the invention what is claimed is:

1. A needle and suture combination comprising:
a. a length of suture material, and
b. a hollow needle with a weakened segment that surrounds and encloses a portion of said suture material which is affixed to said needle a distance from said weaken segment,
whereby said weakened segment of said hollow needle provides a means to separate a portion of said needle with said suture attached.

2. A hollow needle and suture combination in accordance with claim 1 wherein:
said suture material is a monofilament, or
said suture material is multifilament, or
said suture material is braided,
wherein said suture varies in diameter and strength depending on application or procedure.

3. A suture material in accordance with claim 1 wherein:
said suture is a non-absorbable natural fiber material, or
said suture is a non-absorbable synthetic fiber material, or
said suture is a non-absorbable wire material, or
said suture is an absorbable natural material, or
said suture is an absorbable synthetic material.

4. A suture material in accordance with claim 1 wherein:
said suture is composed from a combination of said materials in claim 3.

5. A hollow needle and suture combination in accordance with claim 1 wherein:
said needle is straight, or
said needle is curved, and
said curved needle angle can be manufacture in any number of degrees,
wherein said straight needle and said curved needle length and diameter varies depending on application or procedure.

6. A hollow needle in accordance with claim 1 wherein:
said needle has a weakened segment on said needle outer surface, or
said needle has a weakened segment on said needle inner surface, or
said needle has weakened segments on both said inner surface and said outer surface, and
said needle has multiple said weakened segments along the length of said hollow needle,
which provides a means to vary the length of said portion of said needle when separated.

7. A hollow needle in accordance with claim 1 wherein:
said needle is composed of metal, or
said needle is composed of plastic, or
said needle is composed of a biological absorbable material.

8. A removable needle tip, a hollow needle shaft, and suture combination comprising:
a. a length of suture material, and
b. a separate needle tip having a tapered shaft, and
c. a hollow needle shaft that surrounds and encloses a portion of said suture material and said needle tip tapered shaft which has said suture affixed when assembled,
whereby said needle tip with said suture is removably mounted.

9. A removable needle tip in accordance with claim 8 wherein:
said needle tip provides a means to reattach said suture material, making said needle tip reusable.

10. A hollow needle shaft in accordance with claim 8 wherein:
Said needle shaft has an open end to accept said needle tip tapered shaft with said suture affixed, and
said needle shaft has a closed end,
wherein said needle shaft serves as a reservoir for said suture material.

11. A method of passing suture for use in surgical and non-surgical applications comprising the steps of:
a. providing a hollow needle having an open end and a sharp pointed end, said hollow needle has a weakened segment along said needle shaft, and
b. a length of suture material that enters said open end of hollow needle, travels within and passed said weakened segment where said suture material is then affixed to said suture needle,
whereby said hollow needle and said suture combination is passed through a media until said weakened segment exits said media, and said weakened segment with said suture material attached is separated from said hollow needle providing a means to pass said suture while also allowing withdrawal of remaining said hollow needle from said media leaving said suture material within.

12. A method of passing suture for use in surgical and non-surgical applications comprising the steps in claim 11,
wherein said suture and said needle combination has a needle tip that is removabaly mounted.
